(19) United States
(12) Patent Application Publication
(10) Pub. No.: US 2015/0209192 A1
(43) Pub. Date: Jul. 30, 2015

(54) ENDOTHERMIC SPONGE

(71) Applicant: Empire Technology Development LLC, Wilmington, DE (US)

(72) Inventors: Michael Keoni Manion, Cronulla (AU); Benjamin Matthew Austin, Bangor (AU); George Charles Peppou, Hornsby (AU); William R. Walsh, Maroubra (AU); Nicky Bertollo, Wollstonecraft (AU); Matthew H. Pelletier, Maroubra (AU)

(21) Appl. No.: 14/365,069

(22) PCT Filed: Dec. 16, 2013
(86) PCT No.: PCT/US13/75506

§ 371 Date: Jun. 12, 2014

(51) Int. Cl. A61F 13/36; A61F 13/84; A61F 7/12; A61F 7/02; A61L 31/16; A61L 31/14

(52) U.S. Cl. CPC A61F 13/36; A61L 31/16; A61L 31/146; A61F 7/12; A61F 7/02; A61F 13/8405; A61L 2300/418; A61L 2300/404; A61L 2300/406; A61L 2300/402; A61L 2400/04; A61F 2007/0244; A61F 13/15577

(57) ABSTRACT

Technologies are generally provided for a medical sponge configured to reduce a temperature at a surgical site. Endothermic reactants may be incorporated with the medical sponge to reduce a temperature at the surgical site in response to absorption of fluids. The sponge may be placed at a surgical site, and as the sponge absorbs fluids, the endothermic reactants may become hydrated. Hydration of the endothermic elements may induce an endothermic reaction, resulting in reduction of a temperature of the surrounding surgical site. The endothermic elements may be inserted within semi-permeable pouches that may be layered with the sponge. Additionally, endothermic elements may be inserted within hollow semi-permeable fibers and woven together to form the sponge. Hemostatic agents, antimicrobial agents, and analgesics may also be integrated with the medical sponge to prevent and treat other conditions experienced at the surgical site such as inflammation, pain, and infection.

### BACKGROUND

Unless otherwise indicated herein, the materials described in this section are not prior art to the claims in this application and are not admitted to be prior art by inclusion in this section.

During a surgical procedure, a surgery site may experience an increase in blood flow, and gauze sponges are commonly used to compress the surgical site to absorb the blood and to compress the surgical site to reduce blood flow. Often times, the surgery site can become inflamed and painful during and after the procedure due to increased blood flow, infection, and other causes. Hemostatic agents may be given to help reduce blood flow, and additionally, antimicrobial agents, analgesics and anti-inflammatory medications may be administered to treat symptoms such as inflammation, pain, and infection. However, while the use of various agents may help to treat inflammation and pain symptoms, there may be time delay between the onset of the symptoms and their management.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

According to some examples, the present disclosure describes an absorbent article to reduce a temperature of a site, the article including at least one absorbent portion, and at least one endothermic reactant associated with the absorbent portion, the endothermic reactant configured to induce an endothermic effect in response to a trigger, where the endothermic reactant may be retained within a semi-permeable membrane.

The disclosure also describes a method of forming an absorbent article to absorb fluids from a surgical site and to reduce an ambient temperature of the surgical site. The method may include providing at least one absorbent article, integrating a hemostatic agent with at least a portion of the absorbent article, and integrating at least one endothermic reactant with at least a portion of the absorbent article, the endothermic reactant configured to induce an endothermic effect in response to wetting trigger, wherein the endothermic reactant may be retained within a semi-permeable membrane.

The disclosure further describes a method to absorb fluids from a medical site and reducing an ambient temperature of the medical site, the method may include associating at least one endothermic reactant with an absorbent article, wherein the endothermic reactant may be retained within a semi-permeable membrane and may be configured to induce an endothermic effect in response to a trigger, associating a hemostatic agent with at least a portion of the absorbent article, and inserting the absorbent article including the associated at least one endothermic reactant at a surgical site.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 illustrates an example placement of an endothermic sponge at a surgical site;

FIG. 2 illustrates an example layered endothermic sponge;

FIG. 3 illustrates an example woven endothermic sponge;

FIG. 4 illustrates woven fibers of an endothermic sponge including semi-permeable hollow fibers; and

FIG. 5 illustrates a schematic of example components of an endothermic sponge;

all arranged in accordance with at least some embodiments as described herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, apparatus, systems, devices, and/or computer program products related to providing a medical sponge to reduce a temperature of a surgical site while absorbing fluids from the surgical site.

Briefly stated, technologies are generally provided for a medical sponge configured to reduce a temperature at a medical site. Endothermic reactants may be incorporated with the medical sponge to reduce a temperature at the medical site in response to absorption of fluids. The sponge may be placed at a medical site, and as the sponge absorbs fluids, the endothermic reactants may become hydrated due to becoming wet from exposure to the blood. Wetting and hydration of the endothermic elements may induce an endothermic reaction, resulting in reduction of a temperature of the surrounding medical site. The endothermic elements may be inserted within semi-permeable pouches that may be layered with the sponge. Additionally, endothermic elements may be inserted within hollow semi-permeable fibers and woven together to form the sponge. Hemostatic agents, antimicrobial agents, and analgesics may also be integrated with the medical sponge to prevent and treat other conditions experienced at the medical site such as inflammation, pain, and infection.

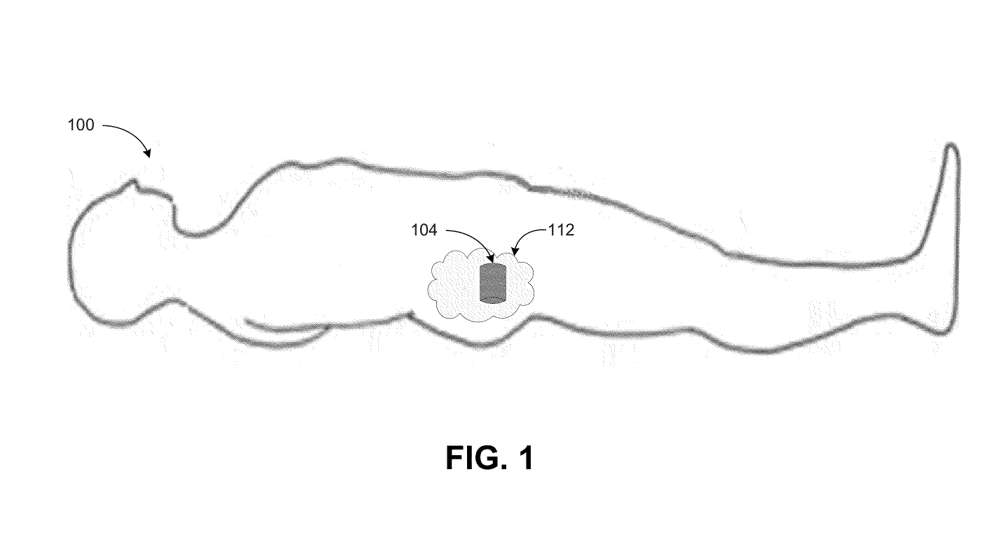

FIG. 1 illustrates an example placement of an endothermic sponge at a medical site, arranged in accordance with at least some embodiments as described herein.

Often times, during surgery, a medical site 112, such as a surgical site where a procedure may be performed on a patient 100, may experience increased blood flow which can cause inflammation and pain during and after the procedure. The medical site 112 may also experience infection due to exposure to microbes and bacteria. Antimicrobial agents, analgesics, and anti-inflammatory medications may be administered to treat inflammation, pain, and infection, however, there may be a lag between the onset of the symptoms and their treatment. Sponges 104 may be placed at the medical site 112 to absorb blood and to induce a hemostatic effect, that is, a reduction of bleeding at the medical site 112.

In an example embodiment, a sponge 104 used to absorb blood at the medical site 112 may incorporate some additional elements to help prevent and treat symptoms experienced at a medical site 112, such as increased bleeding, infection, inflammation and pain. For example, the sponge 104 may incorporate a cooling element such that the sponge may be configured as an endothermic sponge in order to reduce an ambient temperature at the medical site 112 in response to absorption of fluids at the medical site 112. A reduction in temperature at the medical site 112 may cause vasoconstriction of blood vessels which may result in reduced blood flow and thus reduced inflammation and pain at the medical site 112. Cooling the medical site 112 can also reduce pain independent of vaso constriction. Reducing blood flow, inflammation, and pain at the medical site 112 may also improve the effectiveness of post-surgical management of these symptoms.

Additionally, other elements may also be integrated with the medical sponge to prevent and treat other conditions experienced at the medical site 112. Some example elements integrated with the medical sponge may include hemostatic agents, antimicrobial agents, analgesic agents, and anti-inflammatory agents. By incorporating a plurality of preventative elements with the medical sponge, symptoms and conditions commonly experienced at the medical site 112 may be reduced and prevented more effectively without experiencing a time delay between an onset of symptoms and their treatment and management.

While embodiments are described in context of medical applications and surgeries, a sponge as described herein may be employed in fields including, but not limited to critical care, wound management, dentistry, and other non-medical fields where temperature management may be useful.

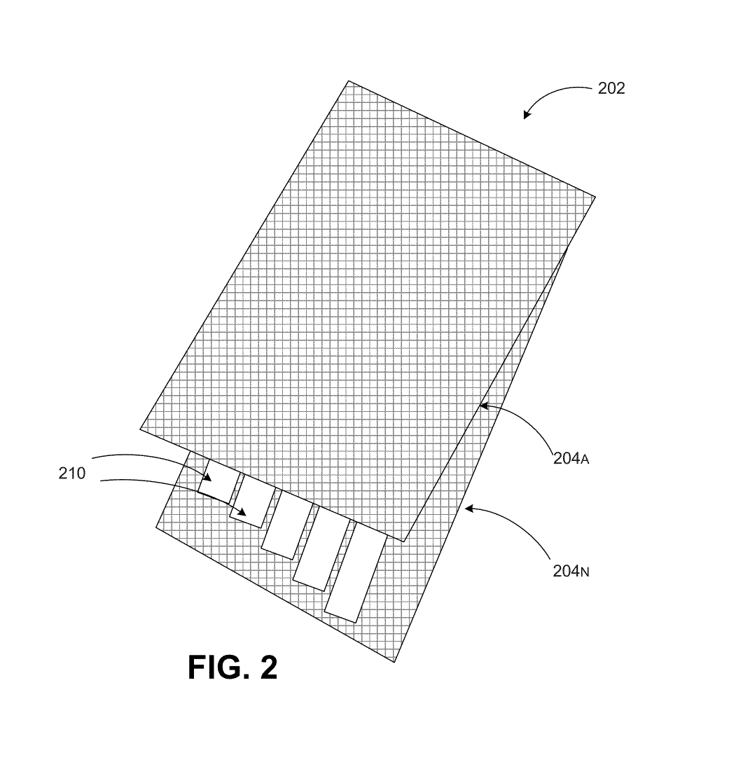

FIG. 2 illustrates an example layered endothermic sponge, arranged in accordance with at least some embodiments as described herein.

As previously described, an absorbent article such as a medical sponge may be configured as an endothermic sponge 202 in order to reduce an ambient temperature at a medical or surgical site to help prevent and treat inflammation by incorporating endothermic elements 210 with the sponge. An example endothermic sponge may formed from gauze, which may be a thin fabric composed from a plurality of fibers having a loose open weave. An endothermic sponge may also be formed from a foam material or a multi-lamellar structure. The sponge may also be formed from a cotton swab, a compress, a pad, and other similar absorbent fabrics.

In a system according to embodiments, one or more cooling or endothermic elements 210 may be layered between two or more layers, such as a top layer204A and a bottom layer 204N of the absorbent article to form the endothermic sponge 202, where the layers may be separately identifiable. The endothermic elements 210 may be configured to undergo an endothermic reaction in response to absorption of fluids, such as blood, causing an endothermic effect, or a reduction in temperature, at the surgical site. An endothermic reaction may be a reaction that absorbs heat from the surrounding environment resulting in a cooling of the surrounding environment. As the sponge absorbs fluid from the surgical site, the endothermic elements 210 may become wet and hydrated, and the hydrated endothermic elements 210 may absorb heat from the surrounding environment to produce a cooling effect on the surrounding environment. In some embodiments, the reaction may also be caused by user activation, which may include mixing the endothermic elements 210 within the pouch, breaking a seal, shaking or other similar method.

In an example embodiment, the endothermic elements 210 may be sealed or retained within one or more pouches, which may be layered between the layers (204A and 204N) of the sponge, such that the endothermic elements 210 may be at least partially sealed within the pouches. The pouches may be composed from a semi-permeable membrane in order to seal the endothermic elements 210 within the pouch and the endothermic sponge 202 while allowing for fluid to penetrate the semi-permeable membrane to cause wetting and hydration of the endothermic elements retained within the pouch. In some examples, the reactant sealed within the pouches may be partially retained by the semi-permeable membrane. For example, the pouch may be partially impermeable and partially semi-permeable. An example membrane may be a polyimide, cellulose, nitrocellulose, or cellophane membrane, or a combination thereof.

Some example endothermic elements 210 that may undergo an endothermic reaction upon wetting and hydration may include one or more inorganic salts having a substantially high heat of hydration. In an example embodiment, the one or more inorganic salts may be retained within the one or more semi-permeable pouches in a dehydrated state. As the sponge absorbs fluid at the surgical site, the fluid may penetrate the pouches, and the inorganic salts may become hydrated. Because of the heat of hydration of the salts, the salts may produce an endothermic effect on the surrounding environment, causing a reduction in the temperature of the surrounding environment. Some example inorganic salts having a substantially high heat of hydration may include potassium nitrate, ammonium nitrate, ammonium chloride, urea, potassium chloride, and potassium sulfate.

Additionally, a hydrophilic compound may be combined with the one or more inorganic salts retained within the semi-permeable pouch. Incorporating the hydrophilic compound may increase absorption of fluid, while also extending the duration of the endothermic effect by increasing a thermal mass within the pouches where the hydration and endothermic reaction may occur. Some example hydrophilic compounds may include a hydrogel, which may be a network of hydrophilic polymer chains, chitosan, hyaluronic acid, alginate, and silicon or a combination thereof.

In another example embodiment, the endothermic elements may be endothermic reactants that result in a hydration reaction and produce an endothermic effect when they become hydrated. For example, a carboxylic acid and a carbonate may react together when they become wet in the presence of blood or other fluids at the medical site, and in response to becoming wet, they may become hydrated. Wetting and hydration of the endothermic reactants from exposure to blood may induce an endothermic reaction, resulting in the reduction of a temperature of the surrounding medical site. Some example carboxylic acids may include citric acid, acetic acid, propanoic acid, tartaric acid, and some example carbonates may include sodium carbonate or calcium carbonate. Other strong acids and bi-carbonates may be employed also. A reaction between a carboxylic acid and a carbonate may also result in gaseous bi-products. In some embodiments, the endothermic sponge may be configured to contain the gaseous bi-products within the sponge so as not to undesirably affect the surrounding surgical site. The contained gaseous bi-products within the sponge may also serve as an insulation layer. In another example embodiment, a rate of the carboxylic acid-carbonate reaction may be controlled in order to cause the reaction to occur slow enough to permit the gas to slowly evacuate the surgical site without producing a negative or undesired effect at the surgical site.

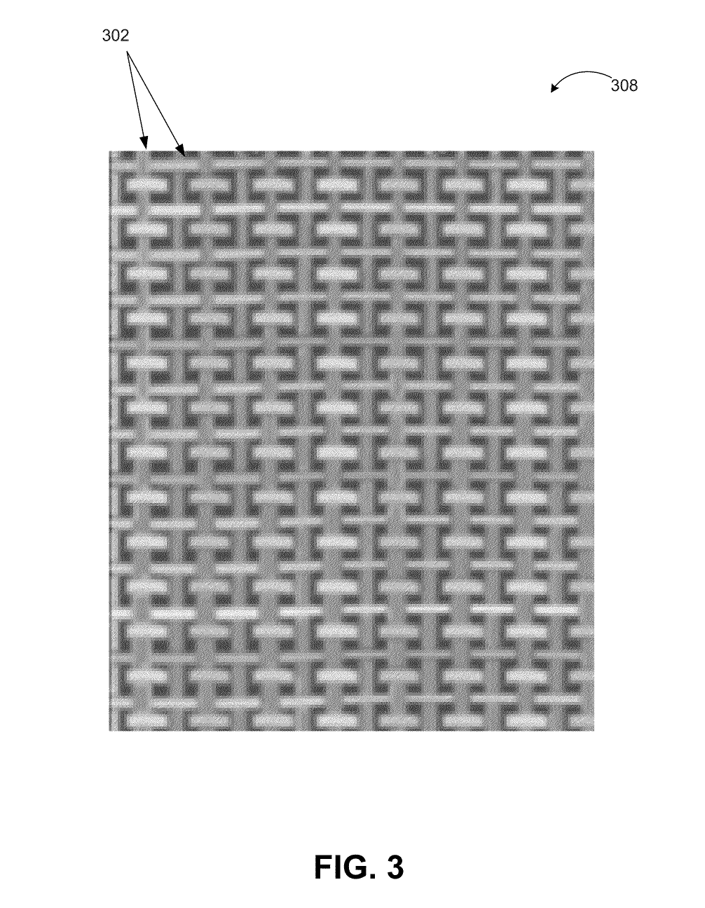

FIG. 3 illustrates an example woven endothermic sponge, arranged in accordance with at least some embodiments as described herein.

As previously described, a medical sponge 308 may be a gauze sponge composed from a plurality of fibers 302 in a loose open weave. The fibers may be woven together employing a braiding technique. The fibers 302 may be composed from cotton and/or other synthetic fibers configured to absorb fluids. The fibers 302 may also be covered with a porous film in order to help prevent direct contact and sticking to minimize adhesion of the sponge with tissue at the surgical site. An example porous film may be as polytetrafluoroethylene (PTFE), which may be a synthetic fluoropolymer of tetrafluoroethylene, or a polyblend. The plurality of fibers 302 may also be coated with an absorbent element in order to increase absorption of fluids and blood at the surgical site. Endothermic elements may be incorporated in between layers of the gauze in order to enable the sponge to induce an endothermic effect on a surrounding environment when placed at a surgical site. In other examples, the endothermic elements may also be incorporated within the woven gauze fibers to produce an endothermic sponge. In other embodiments, the medical sponge may be a foam material or a multi-lamellar structure. The foam material and the multi-lamellar structure may be similarly associated with an absorbent element to increase absorption of fluids and blood at the surgical site.

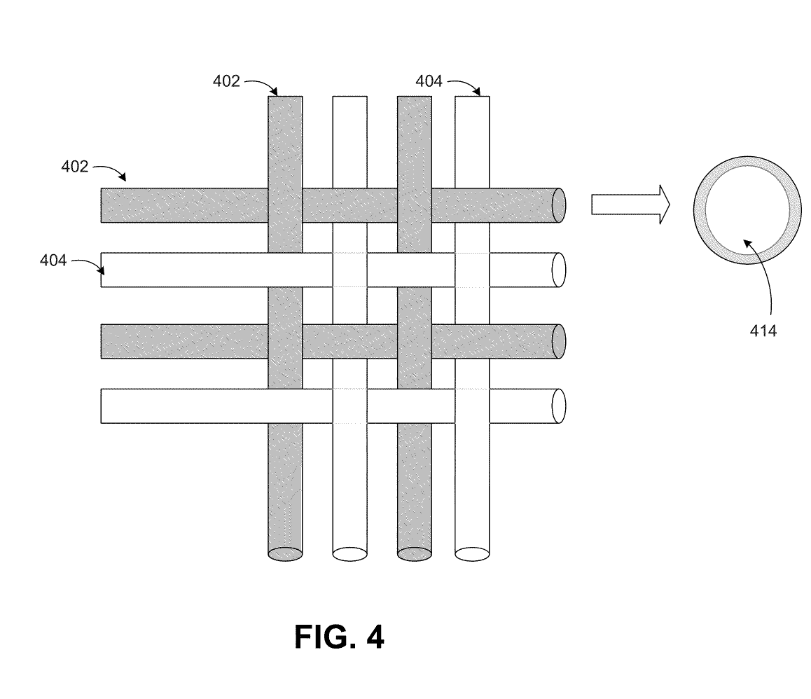

FIG. 4 illustrates woven fibers of an endothermic sponge including semi-permeable hollow fibers, arranged in accordance with at least some embodiments as described herein. As previously described, sponge may be formed from a gauze fabric composed from a plurality of fibers 402, 404 woven together in a flexible and loose open weave. In an example embodiment, a portion of the fibers that may be woven together to form the gauze may be hollow core fibers 402 composed from a semi-permeable membrane. The hollow core fibers 402 may be woven with other cotton and/or synthetic fibers 404 to form the gauze fabric of the endothermic sponge.

In a system according to embodiments, one or more endothermic reactants may be inserted within the hollow core fibers 402, and as the endothermic sponge absorbs fluid at a surgical site, an endothermic reaction may occur within the hollow core fibers 402 behind the semi-permeable membrane. Additionally, the hollow core fibers 402, as well as the cotton and/or synthetic fibers may be coated with additional elements for increasing absorption of fluids and preventing other symptoms at the surgical site such as infection and bleeding. In some additional embodiments, the one or more hollow core fibers 402 may be saturated with the endothermic reactants to incorporate the endothermic reactants with the medical sponge. Furthermore, the endothermic reactants may be selected to be medically safe for the body.

In another example embodiment, the endothermic sponge may be a gauze fabric composed from a plurality of hollow core fibers including one or more endothermic reactants. Each hollow core fiber 402 may be composed from a semi-permeable membrane, and the one or more endothermic reactants may be inserted within the hollow core fibers 402. A cross sectional view 414 of the hollow core fibers illustrates a hollow center into which the one or more endothermic reactants may be inserted. The plurality of hollow core fibers 402 may be treated, sprayed, saturated, or coated with an absorbent element to promote absorption of fluids at the surgical site.

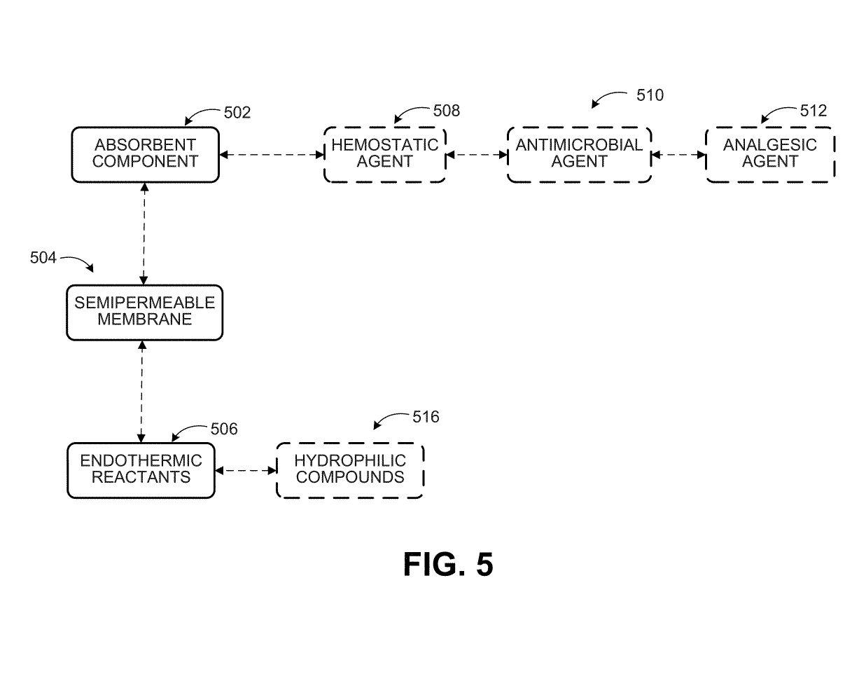

FIG. 5 illustrates a schematic of example components of an endothermic sponge, arranged in accordance with at least some embodiments as described herein.

As previously described, an endothermic sponge may be a gauze sponge composed from a plurality of woven fibers. In some embodiments, the plurality of fibers may include cotton and/or synthetic fibers, and may also include a plurality of hollow core fibers containing one or more endothermic reactants 506. The plurality of hollow core fibers containing the one or more endothermic reactants 506 may be composed from a semi-permeable membrane 504 in order to permit fluids to penetrate the hollow core fibers while containing the endothermic reactants 506 within the hollow core fibers.

In another example embodiment, the endothermic reactants 506 may be retained within one or more pouches composed from the semi-permeable membrane 504, and the semi-permeable pouches may be inserted between layers of the sponge. In another example, the medical sponge may be a foam material or a multi-lamellar structure, and the semi-permeable pouches may be inserted between absorbent layers of the foam material or the multi-lamellar structure that forms the medical sponge.

As fluids are absorbed by the sponge, the fluids may penetrate the semi-permeable membrane to induce an endothermic reaction of the endothermic elements resulting in a reduction in temperature of the surrounding environment. Additionally, other elements, such as hydrophilic compounds may be incorporated with the endothermic reactants 506 retained behind the semi-permeable membrane 504 to increase an endothermic effect. Example hydrophilic compound s 516 may include a hydrogel, chitosan, hyaluronic acid, alginate, and silicone, or a combination thereof.

In a further embodiment, the endothermic sponge may be coated with additional elements to increase fluid absorption, and to prevent and treat other symptoms that may be experienced at the surgical site such as inflammation, infection, and pain. In some embodiments, the plurality of woven fibers may be coated with an absorbent element 502. The absorbent element 502 may help to increase fluid absorption by the sponge, which may also increase hydration of the endothermic reactants resulting in an increased endothermic effect on the surrounding surgical site.

Additionally, a hemostatic agent 508 may be integrated with the woven fibers of the endothermic sponge. The hemostatic agent 508 may reduce bleeding at the surgical site in order to prevent inflammation. In some examples, the hemostatic agent 508 may be integrated with the absorbent element 502 prior to coating the plurality of fibers. Example hemostatic agents may include gelatin, chitosan and fibrinogen, bone wax, mineral zeolite, thrombin, epinephrine, bovine pancreatic trypsin inhibitor (BPTI), phylloquinone, menadione, factor VII, factor IX, carbazochrome, batroxobin, thrombopoietin receptor agonist including one or more of romiplostim and eltrombopag, tetragalacturonic acid hydroxymethylester, calcium alginate, epinephrine, adrenalone, C-1 esterase inhibitor, microfibrillar collagen hemostat (MCH), anhydrous aluminium sulfate, etamsylate, and styptics such as potassium aluminum sulfate, and titanium dioxide, or a combination thereof.

In another embodiment, an antimicrobial agent 510 may also be incorporated with the endothermic sponge in order to help prevent infection at the surgical site. The plurality of woven fibers may be coated with the antimicrobial agent 510. In another embodiment the antimicrobial agent 510 may also be integrated with the absorbent element 502 prior to coating the woven fibers. An example antimicrobial agent 510 may include an antibiotic, a biocide, an antiviral agent, an antiseptic, and an antifungal agent. Furthermore, an analgesic agent 512 may be integrated with the endothermic sponge to help relieve pain at the surgical site. Example analgesic agents may include paracetamol, non-steroidal anti-inflammatory drugs, salicylates, and opioid drugs. The analgesic agent 512 may be incorporated with the absorbent element 502, and may be coated on the plurality of woven fibers of the endothermic sponge.

While embodiments have been discussed above using specific examples, components, and configurations, they are intended to provide a general guideline to be used for an endothermic sponge. These examples do not constitute a limitation on the embodiments, which may be implemented using other components, modules, and configurations using the principles described herein. Furthermore, actions discussed above may be performed in various orders, especially in an interlaced fashion.

According to some examples, the present disclosure describes an absorbent article to reduce a temperature of a site, the article including at least one absorbent portion, and at least one endothermic reactant associated with the absorbent portion, the endothermic reactant configured to induce an endothermic effect in response to a trigger, where the endothermic reactant may be retained within a semi-permeable membrane. The trigger may be one or more of a wetting and a mixing of one or more reactants.

According to some examples, the absorbent portion may include one or more of: a foam material, a multi-lamellar structure, a cotton swab, a compress, a pad, a fabric, and a gauze. The absorbent portion may be formed from woven fibers composed from one or more of: a cotton, a linen, a silk, and a synthetic material.

According to some examples, a hemostatic agent may be integrated with the absorbent element prior to treating. The hemostatic agent may be selected from one or more of: gelatin, chitosan, fibrinogen, bone wax, mineral zeolite, thrombin, epinephrine, bovine pancreatic trypsin inhibitor (BPTI), phylloquinone, menadione, factor VII, factor IX, carbazochrome, batroxobin, thrombopoietin receptor agonist including one or more of romiplostim and eltrombopag, tetragalacturonic acid hydroxymethylester, calcium alginate, epinephrine, adrenalone, C-1 esterase inhibitor, microfibrillar collagen hemostat (MCH), anhydrous aluminium sulfate, potassium aluminum sulfate, titanium dioxide, and etamsylate.

According to other examples, an antimicrobial agent may be integrated with the absorbent element. The antimicrobial agent includes one or more of: an antibiotic, a biocide, an antiviral agent, an antiseptic, and an antifungal agent. An analgesic agent may be integrated with the absorbent element. The at least one endothermic reactant may be sealed within a pouch. The pouch may be composed from a semi-permeable membrane. The semi-permeable membrane may be configured to enable fluids to pass through and configured to inhibit passage of the at least one endothermic reactant.

According to some examples, the semi-permeable membrane may be selected from one or more of: a polyimide, cellulose, nitrocellulose and cellophane membrane. The pouch composed from the semi-permeable membrane may be layered between a first gauze fabric layer and a second gauze fabric layer. A plurality of hollow core fibers may be woven together to form the gauze fabric. The hollow core fiber may be composed from a semi-permeable membrane selected from one or more of: a polyimide, cellulose, nitrocellulose and cellophane membrane.

According to some examples, the at least one endothermic reactant may be inserted within each hollow core fiber in the plurality of hollow core fibers. The plurality of hollow core fibers including the inserted endothermic reactant may be woven along with the woven fibers of the gauze fabric. The plurality of hollow core fibers including the inserted endothermic reactant may be treated with an absorbent element.

According to some examples, the at least one endothermic reactant includes one or more inorganic salts. The inorganic salts may be integrated with the gauze fabric in a dehydrated state. The one or more inorganic salts may be selected from one or more of: potassium nitrate, ammonium nitrate, ammonium chloride, urea, potassium chloride, and potassium sulfate.

According to some examples, a hydrophilic compound may be inserted with the one or more inorganic salts. The hydrophilic compound may be selected from one or more of: a hydrogel, chitosan, hyaluronic acid, alginate, and silicone. The at least one endothermic reactant includes at least one carboxylic acid and at least one carbonate. The gauze fabric may be configured to contain gaseous bi-products of an endothermic reaction between the at least one carboxylic acid and the at least one carbonate.

According to other examples, the present disclosure describes a method of forming an absorbent article to absorb fluids from a surgical site and to reduce an ambient temperature of the surgical site. The method may include providing at least one absorbent article, integrating a hemostatic agent with at least a portion of the absorbent article, and integrating at least one endothermic reactant with at least a portion of the absorbent article, the endothermic reactant configured to induce an endothermic effect in response to wetting trigger, wherein the endothermic reactant may be retained within a semi-permeable membrane. The trigger may be one or more of a wetting and a mixing of one or more reactants.

According to some examples, the absorbent article may include one or more of: a foam material, a multi-lamellar structure, a cotton swab, a compress, a pad, a fabric, and a gauze. The absorbent article may be formed from woven fibers composed from one or more of: a cotton, a linen, a silk, and a synthetic material. The method also includes integrating the hemostatic agent with the absorbent element. The hemostatic agent may be selected from one or more of: gelatin, chitosan, fibrinogen, bone wax, mineral zeolite, thrombin, epinephrine, bovine pancreatic trypsin inhibitor (BPTI), phylloquinone, menadione, factor VII, factor IX, carbazochrome, batroxobin, thrombopoietin receptor agonist including one or more of romiplostim and eltrombopag, tetragalacturonic acid hydroxymethylester, calcium alginate, epinephrine, adrenalone, C-1 esterase inhibitor, microfibrillar collagen hemostat (MCH), anhydrous aluminium sulfate, potassium aluminum sulfate, titanium dioxide, and etamsylate.

According to other examples, the method may also include integrating an antimicrobial agent with the absorbent element. The antimicrobial agent includes one or more of: an antibiotic, a biocide, an antiviral agent, an antiseptic, and an antifungal agent. An analgesic agent may be integrated with the absorbent element. The method may also include sealing the at least one endothermic reactant within a pouch. The method may also include composing the pouch from a semi-permeable membrane. The semi-permeable membrane may be configured to enable fluids to pass through and configured to inhibit passage of the at least one endothermic reactant. The semi-permeable membrane may be selected from one or more of: a polyimide, cellulose, nitrocellulose and cellophane membrane.

According to some examples, the method may also include layering the pouch composed from the semi-permeable membrane between a first gauze fabric layer and a second gauze fabric layer. The method may also include weaving a plurality of hollow core fibers together to form the gauze fabric. The hollow core fiber may be composed from a semi-permeable membrane selected from one or more of: a polyimide, cellulose, nitrocellulose and cellophane membrane.

According to other examples, the method may also include inserting the at least one endothermic reactant within each hollow core fiber in the plurality of hollow core fibers. The method may also include integrating the plurality of hollow core fibers with the woven fibers of the gauze fabric, wherein the plurality of hollow core fibers include the inserted endothermic reactant.

According to some examples, the method may also include treating the plurality of hollow core fibers including the inserted endothermic reactant with an absorbent element. The at least one endothermic reactant includes one or more inorganic salts. The method may also include integrating the inorganic salts with the gauze fabric in a dehydrated state. The one or more inorganic salts may be selected from one or more of: potassium nitrate, ammonium nitrate, ammonium chloride, urea, potassium chloride, and potassium sulfate.

According to some examples, the method may also include inserting a hydrophilic compound with the one or more inorganic salts. The hydrophilic compound may be selected from one or more of: a hydrogel, chitosan, hyaluronic acid, alginate, and silicone. The at least one endothermic reactant includes at least one carboxylic acid and at least one carbonate. The gauze fabric may be configured to contain gaseous bi-products of an endothermic reaction between the at least one carboxylic acid and the at least one carbonate.

According to further examples, the present disclosure describes a method to absorb fluids from a medical site and reducing an ambient temperature of the medical site, the method may include associating at least one endothermic reactant with an absorbent article, wherein the endothermic reactant may be retained within a semi-permeable membrane and may be configured to induce an endothermic effect in response to a trigger, associating a hemostatic agent with at least a portion of the absorbent article, and inserting the absorbent article including the associated at least one endothermic reactant at a surgical site. The trigger may be one or more of a wetting and a mixing of one or more reactants.

According to some examples, the method may also include retaining the at least one endothermic reactant within a pouch composed from a semi-permeable membrane pouch. The method may also include layering the pouch composed from the semi-permeable membrane between a first gauze fabric layer and a second gauze fabric layer. At least a portion of the plurality of woven fibers may be hollow core fibers.

According to some examples, the method may also include inserting the at least one endothermic reactant within each hollow core fiber. The method may also include composing the hollow core fibers from a semi-permeable membrane. The method may also include coating the hollow core fibers with an absorbent element for enabling the gauze fabric to absorb fluids at the surgical site. The at least one endothermic reactant may be an inorganic salt. The method may also include inserting the inorganic salt in a dehydrated form. The method may also include configuring the inorganic salt to become hydrated as fluids may be absorbed by the gauze fabric.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality may be seen as “associated with” each other such that the desired functionality is achieved, irrespective of architectures or intermediate components. Likewise, any two components so associated may also be viewed as being “operably connected,” or “operably “coupled.” to each other to achieve the desired functionality, and any two components capable of being so associated may also be viewed as being “operably couplable,” to each other to achieve the desired functionality. Specific examples of operably couplable include but are not limited to physically connectable and/or physically interacting components and/or wirelessly interactable and/or wirelessly interacting components and/or logically interacting and/or logically interactable components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. An absorbent article to reduce a temperature of a site, the article comprising:
at least one absorbent portion composed from a plurality of hollow core fibers; and
at least one endothermic reactant associated with the absorbent portion such that the plurality of hollow core fibers and the endothermic reactant are woven along with woven fibers of a gauze fabric to form the absorbent portion, the endothermic reactant configured to induce an endothermic effect in response to a trigger, wherein the endothermic reactant is retained within a semi-permeable membrane.

2. The absorbent article of claim 1, wherein the trigger is one or more of a wetting or a mixing of one or more reactants.

3. The absorbent article of claim 1, wherein the absorbent portion includes one or more of: a foam material, a multi-lamellar structure, a cotton swab, a compress, a pad, a fabric, and a gauze.

4. (canceled)

5. The absorbent article of claim 1, wherein the woven fibers are treated with an absorbent substance.

6. The absorbent article of claim 5, wherein a hemostatic agent is associated with the absorbent substance prior to coating.

7. The absorbent article of claim 6, wherein the hemostatic agent is selected from one or more of: gelatin, chitosan, fibrinogen, bone wax, mineral zeolite, thrombin, epinephrine, bovine pancreatic trypsin inhibitor (BPTI), phylloquinone, menadione, factor VII, factor IX, carbazochrome, batroxobin, thrombopoietin receptor agonist including one or more of romiplostim and eltrombopag, tetragalacturonic acid hydroxymethylester, calcium alginate, epinephrine, adrenalone, C-1 esterase inhibitor, microfibrillar collagen hemostat (MCH), anhydrous aluminium sulfate, potassium aluminum sulfate, titanium dioxide, and etamsylate.

8. The absorbent article of claim 1, further comprising an antimicrobial agent, wherein the antimicrobial agent includes one or more of: an antibiotic, a biocide, an antiviral agent, an antiseptic, and an antifungal agent.

9. (canceled)

10. The absorbent article of claim 1, further comprising an analgesic agent.

11.-15. (canceled)

16. The absorbent article of claim 1, wherein the at least one endothermic reactant is inserted within each hollow core fiber in the plurality of hollow core fibers.

17.-25. (canceled)

26. A method of forming an absorbent article to absorb fluids from a surgical site and to reduce an ambient temperature of the surgical site, comprising:
providing at least one absorbent article composed from a plurality of hollow core fibers;
integrating a hemostatic agent with at least a portion of the absorbent article; and
integrating at least one endothermic reactant with at least a portion of the absorbent article such that the plurality of hollow core fibers and the endothermic reactant are woven along with woven fibers of a gauze fabric to form an absorbent portion, the endothermic reactant configured to induce an endothermic effect in response to wetting trigger, wherein the endothermic reactant is retained within a semi-permeable membrane.

27.-35. (canceled)

36. The method of claim 26, further comprising:
retaining the at least one endothermic reactant within a pouch, wherein the pouch is composed from semi-permeable membrane.

37. (canceled)

38. The method of claim 36, wherein the semi-permeable membrane is configured to enable fluids to pass through and configured to inhibit passage of the at least one endothermic reactant.

39. The method of claim 36, further comprising:
layering the pouch composed from the semi-permeable membrane between a first absorbent layer and a second absorbent layer of the absorbent article.

40. (canceled)

41. The method of claim 26, further comprising:
inserting the at least one endothermic reactant within each hollow core fiber in the plurality of hollow core fibers.

42. (canceled)

43. The method of claim 41, further comprising:
coating the plurality of hollow core fibers including the at least one inserted endothermic reactant with an absorbent element.

44. The method of claim 26, wherein the at least one endothermic reactant includes one or more inorganic salts.

45. The method of claim 44, further comprising:
integrating the inorganic salts with the absorbent article in a dehydrated state, wherein the one or more inorganic salts are selected from the group consisting of: potassium nitrate, ammonium nitrate, ammonium chloride, urea, potassium chloride, and potassium sulfate.

46. (canceled)

47. The method of claim 44, further comprising:
inserting a hydrophilic compound with the one or more inorganic salts, wherein the hydrophilic compound is selected from the group consisting of: a hydrogel, chitosan, hyaluronic acid, alginate, and silicone.

48. (canceled)

49. The method of claim 26, wherein the at least one endothermic reactant includes at least one carboxylic acid and at least one carbonate.

50. The method of claim 49, wherein the absorbent article is configured to contain gaseous bi-products of an endothermic reaction between the at least one carboxylic acid and the at least one carbonate.

51. A method to absorb fluids from a medical site and reducing an ambient temperature of the medical site, the method comprising:
associating at least one endothermic reactant with an absorbent article such that a plurality of hollow core fibers composing the absorbent article and the endothermic reactant are woven along with woven fibers of a gauze fabric to form an absorbent portion, wherein the endothermic reactant is retained within a semi-permeable membrane, and is configured to induce an endothermic effect in response to a trigger,
associating a hemostatic agent with at least a portion of the absorbent article; and
inserting the absorbent article including the associated at least one endothermic reactant at a surgical site.

52.-55. (canceled)

56. The method of claim 51, wherein the absorbent article is formed from woven fibers composed from one or more of: a cotton, a linen, a silk, and a synthetic material.

57.-58. (canceled)

59. The method of claim 56, further comprising:
treating the hollow core fibers with an absorbent substance to enable the absorbent article to absorb fluids at the surgical site.

60.-62. (canceled)
